(19) United States
(12) Patent Application Publication
(10) Pub. No.: US 2023/0218430 A1
(43) Pub. Date: Jul. 13, 2023

(54) CORONAVIRUS-KILLING AND SCALP CARE DEVICE USING CARBON HEATING MAT

(71) Applicant: Billian Ducwong HYUN, Artesia, CA (US)

(72) Inventor: Billian Ducwong HYUN, Artesia, CA (US)

(21) Appl. No.: 17/672,973

(22) Filed: Feb. 16, 2022

(30) Foreign Application Priority Data
Jan. 13, 2022 (KR) 10-2022-0005305

(51) Int. Cl. A61F 7/00; A61L 9/04

(52) U.S. Cl. CPC A61F 7/007; A61L 9/04; A61F 2007/0008; A61H 2205/021; A61F 2007/0003; A61M 2021/0016

(57) ABSTRACT

Provided is a coronavirus-killing and scalp care device using a carbon heating mat. Carbon heating mats are applied to a coronavirus-killing face cover and a scalp cover which are coupled to or separated from each other as necessary, and a filter part for accommodating a scent material is formed around a nose and mouth portion of the coronavirus-killing face cover. Therefore, a temperature of the scalp is raised to boost immunity, prevent hair loss, and the like, heat is introduced into the human body during breathing through the nose and mouth to kill various disease-causing pathogens (particularly, coronaviruses) in the human body, and a scent material filling the filter part is fumigated by heat generated by the carbon heating mats and introduced into the human body through breathing to prevent and cure diseases.

### BACKGROUND

### 1. Field of the Invention

The present invention relates to a coronavirus-killing and scalp care device using a carbon heating mat which kills coronavirus and cares for the scalp using the carbon heating mat without electromagnetic waves.

### 2. Discussion of Related Art

Currently, there is no technology or drug which kills the so-called coronavirus. According to Factman's experiment, when the coronavirus is heated at 60° C. for an hour, among three million coronaviruses, only five coronaviruses survive.

Humans can survive an hour at 60° C. Accordingly, the coronavirus infecting the human body can be killed by performing heating twice for an hour each.

The coronavirus can be killed by wearing a heating mask using a carbon heating mat on the face and inhaling heat of 60° C. The effect is quicker when oregano oil and eucalyptus oil, which are aromatic oils, are used in addition to the heat.

In addition, a scalp care device may also obtain effects of sterilizing the scalp and preventing hair loss.

### SUMMARY OF THE INVENTION

The present invention is directed to providing a coronavirus-killing and scalp care device using a carbon heating mat in which the carbon heating mat is applied to each of a coronavirus-killing face cover and a scalp cover which are coupled to or separated from each other as necessary to raise a temperature of the scalp in order to boost immunity and prevent hair loss, and the like and which allows heat to be introduced into the human body during breathing through the nose and mouth to kill various disease-causing pathogens (particularly, coronaviruses) in the human body.

The present invention is also directed to providing a coronavirus-killing and scalp care device using a carbon heating mat in which a filter part for accommodating a scent material is formed around a nose and mouth portion of a coronavirus-killing face cover and various scent materials filling the filter part are fumigated by heat generated by a carbon heating mat and are introduced into the human body through breathing to prevent and cure diseases.

According to an aspect of the present invention, there is provided a coronavirus-killing and scalp care device using a carbon heating mat, including a coronavirus-killing face cover which includes a first wearable band, on which female and male latching parts are formed to fix the coronavirus-killing face cover when the coronavirus-killing face cover is worn on a face of a wearer, and one or more ventilation portions formed in a nose and mouth portion, a scalp cover which includes a second wearable band for fixing the scalp cover when the scalp cover is worn on the head and is detachably coupled to the coronavirus-killing face cover, a carbon heating mat installed in each of the coronavirus-killing face cover and the scalp cover and heated by applied driving power, and a control panel through which the driving power is supplied to the carbon heating mats.

A first coupling part having a Velcro type may be formed on an outer circumferential surface of an upper end of the coronavirus-killing face cover, and a second coupling part having a Velcro type corresponding to the first coupling part may be formed on one end of the scalp cover.

A rubber film, which is in contact with skin to block internal heat of the coronavirus-killing face cover from being discharged to an outside when the coronavirus-killing face cover is worn on the face, may be formed on an edge side of the coronavirus-killing face cover.

The carbon heating mat installed in the coronavirus-killing face cover may be covered by a first inner cover.

A chin support part for supporting the chin of the wearer may be on a lower surface of the coronavirus-killing face cover.

The carbon heating mat installed in the scalp cover may be covered by a second inner cover.

A third coupling part having a Velcro type may be formed on an inner edge surface of the scalp cover, and a fourth coupling part having a Velcro type corresponding to the third coupling part may be formed on an outer edge surface of the second inner cover.

Breath members and a scent member may each be coupled to the ventilation portions.

The breath member may include a breathing cylinder part coupled to the plurality of ventilation portions, a first filter membrane formed on an opening in one end of the breathing cylinder part, and a first cover part detachably coupled to an opening in the other end of the breathing cylinder part, and a second filter membrane may be formed on the first cover part.

The scent member may include a scent container which is coupled to the ventilation portion disposed between the breath members and in which a scent material is accommodated and a second cover part coupled to an opening in an upper end of the scent container, wherein a third filter membrane may be formed on the second cover part.

The scent material accommodated in the scent container may be a mixture of oregano oil and eucalyptus oil for aromatherapy.

Power lines to be connected to the carbon heating mats may be led from the coronavirus-killing face cover and the scalp cover, the led power lines may be connected to the control panel having a power cord, and a plurality of operation switches, which turn heating of the carbon heating mats on or off, and a plurality of temperature adjustment switches, which adjust an increase/decrease in heating temperature of the carbon heating mat, may be formed on the control panel.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a combined perspective view illustrating a care device according to an embodiment of the present invention;

FIG. 2 is an exploded perspective view illustrating a care device according to an embodiment of the present invention;

FIG. 3 is a configuration diagram illustrating a control panel connected to a coronavirus-killing face cover and a scalp cover through a power line according to an embodiment of the present invention;

FIG. 4 is a perspective rear view illustrating a coronavirus-killing face cover according to an embodiment of the present invention.

FIG. 5 is schematic cross-sectional view illustrating a coronavirus-killing face cover according to an embodiment of the present invention;

FIG. 6 is schematic cross-sectional view illustrating a scalp cover according to an embodiment of the present invention; and

FIG. 7 is a view illustrating a state in which a care device is worn according to an embodiment of the present invention.

### DETAILED DESCRIPTION OF EXEMPLARY EMBODIMENTS

Hereinafter, embodiments of the present invention will be described with reference to the accompanying drawings.

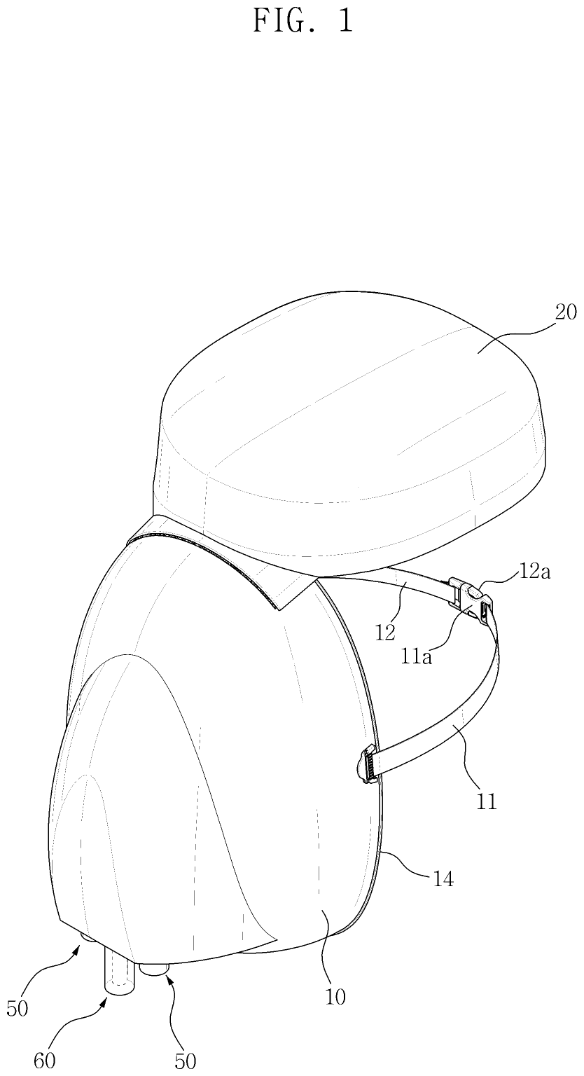

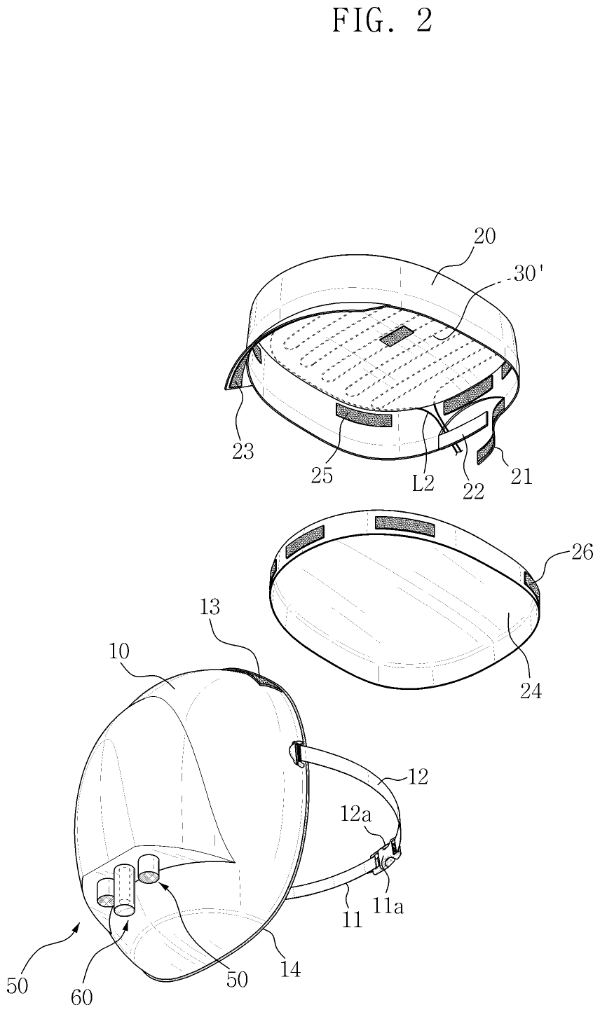

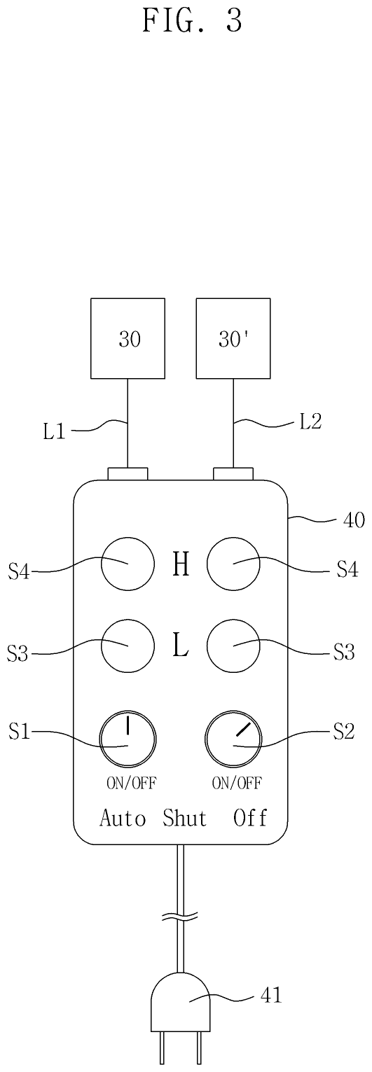

FIG. 1 is a combined perspective view illustrating a care device according to an embodiment of the present invention, FIG. 2 is an exploded perspective view illustrating the care device according to the embodiment of the present invention, and FIG. 3 is a configuration diagram illustrating a control panel connected to a coronavirus-killing face cover and a scalp cover through a power line according to the embodiment of the present invention.

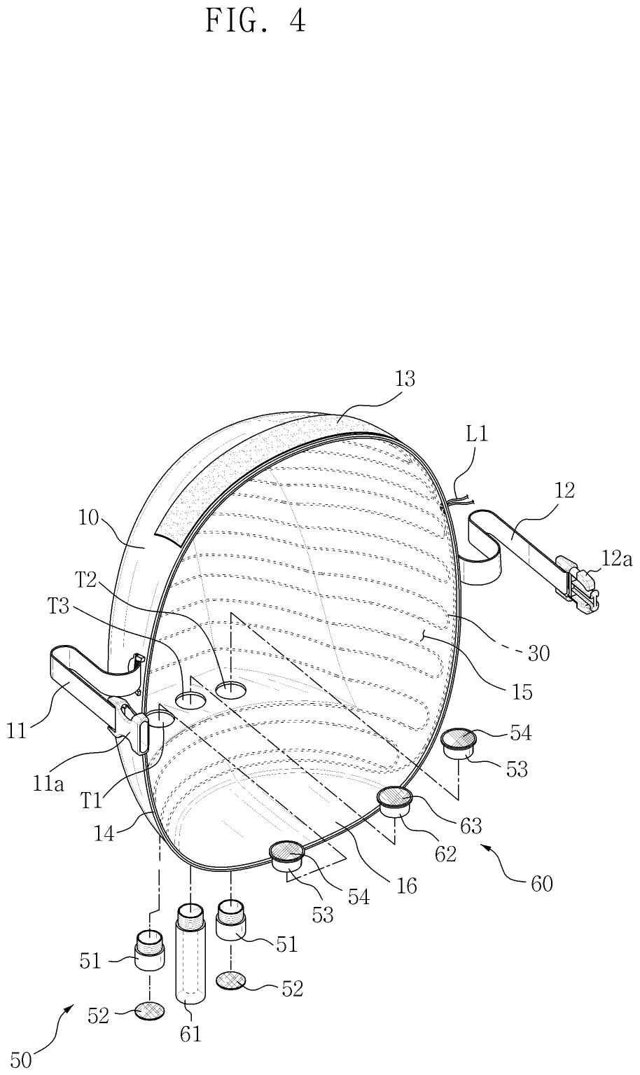

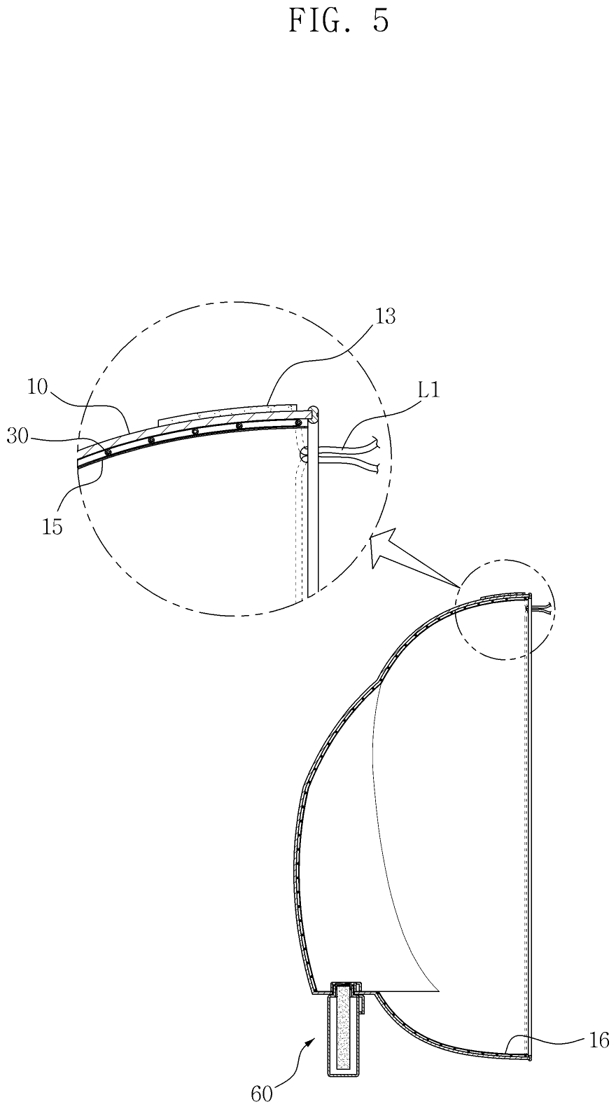

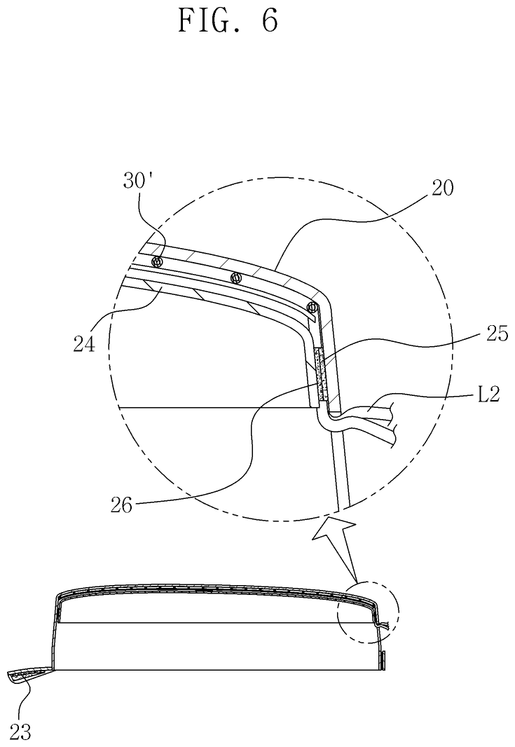

FIG. 4 is a perspective rear view illustrating the coronavirus-killing face cover according to the embodiment of the present invention, FIG. 5 is schematic cross-sectional view illustrating the coronavirus-killing face cover according to the embodiment of the present invention, and FIG. 6 is schematic cross-sectional view illustrating the scalp cover according to the embodiment of the present invention.

Referring to the accompanying FIGS. 1 to 6, a coronavirus-killing and scalp care device using a carbon heating mat according to the embodiment of the present invention may include a coronavirus-killing face cover 10, a scalp cover 20, carbon heating mats 30 and 30′, a control panel 40, and/or a breath member 50 and a scent member 60.

As illustrated in the accompanying FIGS. 1, 2, 4, and 5, the coronavirus-killing face cover 10 covers the whole face of a wearer and includes first wearable bands 11 and 12 at which locking parts 11a and 12a are formed to fix the coronavirus-killing face cover 10 to the face in a female and male hooking manner when worn on the face, one or more ventilation portions T1, T2, and T3 may be formed in a nose and mouth portion, and the first wearable bands 11 and 12 surround a rear side of a head of the wearer.

In addition, a first coupling part 13 having a Velcro type is formed on an outer circumferential surface of an upper end of the coronavirus-killing face cover 10, a rubber film 14, which is in contact with skin to block internal heat of the coronavirus-killing face cover 10 from being discharged to the outside when the coronavirus-killing face cover 10 is worn on the face, may also be formed on an edge side of the coronavirus-killing face cover 10, a first inner cover 15 for covering the carbon heating mat 30 installed in the coronavirus-killing face cover 10 may also be formed, and a chin support part 16 for supporting the chin of the wearer may also be formed on a lower surface of the coronavirus-killing face cover 10.

The scalp cover 20 includes second wearable bands 21 and 22 having a Velcro type for being fixed to the head when the scalp cover 20 is worn on the head and may be detachably coupled to the coronavirus-killing face cover 10 as illustrated in the accompanying FIGS. 1, 2, and 6.

That is, as a second coupling part 23 having a Velcro type is formed at one end of the scalp cover 20, the second coupling part 23 is coupled to the first coupling part 13 formed on the outer circumferential surface of the upper end of the coronavirus-killing face cover 10, the scalp cover 20 may be coupled to the coronavirus-killing face cover 10, and when the coupling between the first and second coupling parts 13 and 23 is released, the scalp cover 20 may be separated from the coronavirus-killing face cover 10.

In this case, a second inner cover 24 for covering the carbon heating mat 30′ installed in the scalp cover 20 may be formed thereon, the second inner cover 24 is a fabric which is attachable to and detachable from the scalp cover 20 and washable, a third coupling part 25 having a Velcro type may be accordingly formed on an inner edge surface of the scalp cover 20, and one or more fourth coupling parts 26 having a Velcro type and disposed to correspond to the third coupling part 25 may be formed on an outer edge surface of the second inner cover 24.

In this case, a length of each of the second wearable bands 21 and 22 may be adjusted according to a circumference of the head of the wearer.

The carbon heating mats 30 and 30′ are respectively installed in the coronavirus-killing face cover 10 and the scalp cover 20, covered by the first and second inner covers 15 and 24, and heated by applied driving power.

That is, each of the carbon heating mats 30 and 30′ is a known surface heater using carbon fiber thread manufactured by coating or impregnating thread having heat resistance with carbon fiber, and when power is applied to the carbon fiber thread, the carbon fiber serves as a resistor to generate resistance heat so that the carbon heating mats 30 and 30′ may be used as heaters.

Accordingly, in each of the carbon heating mats 30 and 30′ which are the surface heater woven using the carbon fiber thread, a temperature may be easily raised in a short time due to high electrical resistance of the carbon fiber thread, and electromagnetic waves are not generated. Since each of the carbon heating mats 30 and 30′ is known to have a constant temperature characteristic that a temperature does not rise further when a predetermined temperature is reached, power consumption is minimized, and thus there is no burn risk. In addition, in the embodiment of the present invention, a known carbon heating mat may be applied to the care device.

The control panel 40 allows the carbon heating mats 30 and 30′ to receive the driving power, and thus power lines L1 and L2 respectively connected to the carbon heating mats 30 and 30′ may be led to the coronavirus-killing face cover 10 and the scalp cover 20. The led power lines L1 and L2 may be connected to the control panel 40 having a power cord 41, and as illustrated in the accompanying FIG. 3, a plurality of operation switches S1 and S2 which turn heating of the carbon heating mats 30 and 30′ on or off and a plurality of temperature adjustment switches S3 and S4 which adjust heating temperatures of the carbon heating mats 30 and 30′ up or down may be formed on the control panel 40.

The breath member 50 is coupled to a plurality of ventilation portions T1 and T2 formed in the coronavirus-killing face cover 10 and includes breathing cylinder parts 51, first filter membranes 52, first cover parts 53, and second filter membranes 54 as illustrated in the accompanying FIG. 4.

The breathing cylinder parts 51 may be coupled to the ventilation portions T1 and T2, the first filter membranes 52 may be formed on openings in one ends of the breathing cylinder parts 51, the first cover parts 53 may be detachably coupled to openings in the other ends of the breathing cylinder parts 51, and the second filter membranes 54 may be formed on the first cover parts 53.

Accordingly, the first and second filter membranes 52 and 54 formed on both ends of the breathing cylinder part 51 may allow the wearer to breath smoothly and block foreign matters from being introduced through the breathing.

The scent member 60 is coupled to the other ventilation portion T3 formed between the plurality of ventilation portions T1 and T2 formed in the coronavirus-killing face cover 10 and may include a scent container 61, a second cover part 62, and a third filter membrane 63 as illustrated in the accompanying FIG. 4.

The scent container 61 is coupled to the ventilation portion T3, and a scent material may be accommodated in the scent container 61.

The second cover part 62 is coupled to an opening in an upper end of the scent container 61, and the third filter membrane 63 may be formed on the second cover part 62.

In this case, the scent material accommodated in the scent container 61 may be a mixture of oregano oil and eucalyptus oil for aromatherapy which is known to have a pharmacological effect of neutralizing toxicity, but is not limited thereto.

Accordingly, when the wearer breathes in a state in which the whole face of the wearer is covered by the coronavirus-killing face cover 10, the scent emanating from the scent container 61 may be fumigated by heat of the carbon heating mats 30 and 30′ and inhaled.

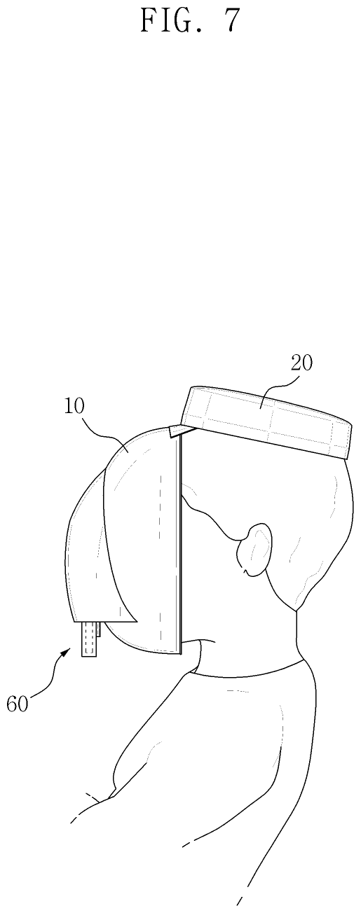

As described above, in the coronavirus-killing and scalp care device using a carbon heating mat according to the embodiment of the present invention, first, in a state in which the power cord 41 of the control panel 40 is plugged into a power outlet and the coronavirus-killing face cover 10 is coupled to the scalp cover 20, the coronavirus-killing face cover 10 is worn to cover the whole face using the first wearable bands 11 and 12, and the scalp cover 20 is worn to cover the head using the second wearable bands 21 and 22 as illustrated in the accompanying FIGS. 1 to 7.

In this case, the scent material should be contained in the scent container 61 formed in the coronavirus-killing face cover 10, but the scent material may not be contained therein.

Then, the plurality of operation switches S1 and S2 provided on the control panel 40 are switched on to apply power to the carbon heating mats 30 and 30′ installed in the coronavirus-killing face cover 10 and the scalp cover 20 through the power lines L1 and L2.

Accordingly, while the carbon heating mats 30 and 30′ are heated, the heat generated by the carbon heating mats 30 and 30′ may be transferred to the skin of the face and the scalp through the first inner cover 15 and the second inner cover 24.

In this case, when the heat is transferred to the skin of the face of the wearer through the first inner cover 15, the heat may be introduced into the human body through the nose or mouth during breathing, and in this case, the scent emanating from the scent container 61 is fumigated, and the fumigated scent may be introduced into the human body.

In this case, temperatures of the heat generated by the carbon heating mats 30 and 30′ may be adjusted using the plurality of temperature adjustment switches S3 and S4 to increase or decrease the temperatures, the temperature of the carbon heating mat 30 installed in the coronavirus-killing face cover 10 is controlled in the range of 38° C. to 75° C. by the temperature adjustment switch S3, and the temperature of the carbon heating mat 30′ installed in the scalp cover 20 is controlled in the range of 40° C. to 75° C. by the temperature adjustment switch S4, but the present invention is not limited such temperatures.

Meanwhile, the scent container 61 included in the scent member 60 is fixedly coupled to the coronavirus-killing face cover 10, and since the mixture of the oregano oil and the eucalyptus oil, which are scent materials for aromatherapy, is accommodated in the scent container 61, when the carbon heating mat 30 installed in the coronavirus-killing face cover 10 is heated, while the scent material is fumigated, a fumigated component of the scent material may be introduced into the body of the wearer wearing the coronavirus-killing face cover 10, and thus various disease-causing pathogens in the human body may be killed.

In addition, due to the heat of carbon heating mats 30 and 30′ installed in the coronavirus-killing face cover 10 and the scalp cover 20, blood circulation in the skin of the face and the scalp is promoted, sweat is discharged from the skin, waste in pores is removed, and cell generation of the face skin is promoted so that wrinkles may be removed, a whitening effect may be obtained, and skin aging may also be prevented.

That is, in the care device according to the embodiment of the present invention, in a state in which the coronavirus-killing face cover 10 is worn on the face and the scalp cover 20 is worn on the head, when the coronavirus-killing face cover 10 and the scalp cover 20 are repeatedly electrically heated using the carbon heating mats 30 and 30′ several times (for example, two times), for a predetermined time (for example, an hour) each, the coronavirus can be killed as in the result of Factman's experiment in which only five coronaviruses survived among three million coronaviruses when 60° C. heat was applied to the coronaviruses for an hour, immunity can also be boosted, and hair loss can also be prevented by raising a temperature of the scalp. Particularly, when a user breathes the fumigated scent material through the nose and mouth, an effect of killing the coronavirus can be improved further.

As described above, in the present invention, a carbon heating mat is applied to each of a coronavirus-killing face cover and a scalp cover which are coupled to or separated from each other as necessary, and a filter part for accommodating a scent material is formed around a nose and mouth portion of the coronavirus-killing face cover. Therefore, effects that can be expected include a temperature of the scalp being raised to boost immunity, prevent hair loss, and the like, heat being introduced into the human body during breathing through the nose and mouth to kill various disease-causing pathogens (particularly, coronaviruses) in the human body, and scent materials with which the filter part is filled being fumigated by heat generated by the carbon heating mat and introduced into the human body through breathing to prevent and cure diseases.

While the technical spirit of the coronavirus-killing and scalp care device using a carbon heating mat of the present invention has been described with reference to the accompanying drawings, this is only an illustrative description of the exemplary embodiments of the present invention, and does not limit the present invention.

Accordingly, the present invention is not limited to the above-described specific exemplary embodiments and may be variously modified by those skilled in the art without departing from the gist of the invention claimed by the appended claims, and the modifications are within the scope of the claims.

What is claimed is:

1. A coronavirus-killing and scalp care device using a carbon heating mat, comprising:
a coronavirus-killing face cover which includes a first wearable band, on which female and male latching parts are formed to fix the coronavirus-killing face cover when the coronavirus-killing face cover is worn on a face of a wearer, and one or more ventilation portions formed in a nose and mouth portion;
a scalp cover which includes a second wearable band for fixing the scalp cover when the scalp cover is worn on the head and is detachably coupled to the coronavirus-killing face cover;
a carbon heating mat installed in each of the coronavirus-killing face cover and the scalp cover and heated by applied driving power; and
a control panel through which the driving power is supplied to the carbon heating mat.

2. The coronavirus-killing and scalp care device using a carbon heating mat of claim 1, wherein:
a first coupling part having a Velcro type is formed on an outer circumferential surface of an upper end of the coronavirus-killing face cover; and
a second coupling part having a Velcro type corresponding to the first coupling part is formed on one end of the scalp cover.

3. The coronavirus-killing and scalp care device using a carbon heating mat of claim 1, wherein a rubber film, which is in contact with skin to block internal heat of the coronavirus-killing face cover from being discharged to an outside when the coronavirus-killing face cover is worn on the face, is formed on an edge side of the coronavirus-killing face cover.

4. The coronavirus-killing and scalp care device using a carbon heating mat of claim 1, wherein the carbon heating mat installed in the coronavirus-killing face cover is covered by a first inner cover.

5. The coronavirus-killing and scalp care device using a carbon heating mat of claim 1, wherein the carbon heating mat installed in the scalp cover is covered by a second inner cover.

6. The coronavirus-killing and scalp care device using a carbon heating mat of claim 5, wherein:
a third coupling part having a Velcro type is formed on an inner edge surface of the scalp cover; and
a fourth coupling part having a Velcro type corresponding to the third coupling part is formed on an outer edge surface of the second inner cover.

7. The coronavirus-killing and scalp care device using a carbon heating mat of claim 1, wherein:
breath members and a scent member are each coupled to the ventilation portions;
the breath member includes a breathing cylinder part coupled to the plurality of ventilation portions, a first filter membrane formed on an opening in one end of the breathing cylinder part, and a first cover part detachably coupled to an opening in the other end of the breathing cylinder part; and
a second filter membrane is formed on the first cover part.

8. The coronavirus-killing and scalp care device using a carbon heating mat of claim 7, wherein:
the scent member includes a scent container which is coupled to the ventilation portion disposed between the breath members and in which a scent material is accommodated; and
a second cover part coupled to an opening in an upper end of the scent container,
wherein a third filter membrane is formed on the second cover part.

9. The coronavirus-killing and scalp care device using a carbon heating mat of claim 8, wherein the scent material accommodated in the scent container includes a mixture of oregano oil and eucalyptus oil for aromatherapy.

10. The coronavirus-killing and scalp care device using a carbon heating mat of claim 1, wherein:
power lines to be connected to the carbon heating mats are led from the coronavirus-killing face cover and the scalp cover;
the led power lines are connected to the control panel having a power cord; and
a plurality of operation switches, which turn heating of the carbon heating mats on or off, and a plurality of temperature adjustment switches, which adjust an increase/decrease in heating temperature of the carbon heating mat, are formed on the control panel.
